(19) United States
(12) Patent Application Publication
(10) Pub. No.: US 2002/0137663 A1
(43) Pub. Date: Sep. 26, 2002

(54) The anti-neoplastic agent ET-743 inhibits trans activation by SXR

(76) Inventors: Barry M. Forman, Newport Beach, CA (US); Isabelle Dussault, Thousand Oaks, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 09/927,341

(22) Filed: Aug. 13, 2001

Related U.S. Application Data
(60) Provisional application No. 60/224,356, filed on Aug. 11, 2000.

(51) Int. Cl. A61K 31/00

(52) U.S. Cl. 514/1

(57) ABSTRACT

ET-743 is a small molecular weight compound with antineoplastic activity that inhibits the ability of the nuclear receptor SXR to trans activate gene transcription from SXR regulated response elements. The nuclear receptor SXR has been identified as a receptor that activates transcription of the mdr1 gene and thus increases multidrug resistance in cells. The interaction of SXR with the mdr1 gene and ET-743 provide a set of physiological mechanisms which can be exploited to identify novel inhibitors of SXR activation and mdr1 gene transcription and thus novel agents which exhibit an antineoplastic effect against tumor cells either alone or when coadministered with another antineoplastic agent.

### CROSS-REFERENCE TO RELATED APPLICATIONS

This application is related to provisional patent application Ser. No. 60/224,356, filed Aug. 11, 2000, and claims the benefit of the filing date of said provisional application.

### BACKGROUND OF THE INVENTION

The present invention is directed to methods of screening compounds for anti-neoplastic activity. The invention is also directed to compounds that inhibit trans activation of target gene transcription by the SXR nuclear receptor and methods for the detection of such compounds.

The publications and other materials used herein to illuminate the background of the invention or provide additional details respecting the practice, are incorporated by reference, and for convenience are respectively grouped in the appended List of References.

Ecteinascidin-743 (ET-743, NSC 648766) is a novel, low molecular weight, anti-neoplastic drug that holds considerable promise for clinical used¹. It is currently in Phase II trials and has been proposed for clinical evaluation against a variety of tumors including melanoma, breast, non-small-cell lung, and ovarian cancers^{2,3}. Of particular note is that the drug is active against sarcomas which generally lack alternative chemotherapeutic options. ET-743 possesses extremely potent cytotoxic activity; it inhibits the growth of a variety of cancer cell-lines and human xenografts with IC₅₀s ranging from 1-100 nM^{2,4}. This range of cytotoxic activity is 10-1000 fold more potent than some of the more common chemotherapeutic agents including taxol, camptothecin, adriamycin, mitomycin, cisplatin, bleomycin and etoposide. The high potency of ET-743 implies that it acts through a specific molecular target.

Despite its considerable promise, the mechanism by which ET-743 induces its cytotoxic response has not been established to date. ET-743 has been reported to promote a variety of interesting activities. These include: binding to DNA in the minor groove⁵, alkylation of guanines at the N2 position⁶ and promotion of topoisomerase I-mediated cross-linking to DNA breaks^{4,7}. ET-743 has also been shown to inhibit DNA binding by the NF-Y transcription factor^{8,9}. It remains unclear if any of these phenomena are related to the cytotoxic effects of ET-743, as they are all induced at micromolar concentrations whereas the cytotoxic effect of the drug is clearly evident in the low nanomolar range.

One of the genes responsible for multi-drug resistance to chemotherapy is mdr1 which encodes a protein that is variously called P-glycoprotein, Pgp or P170, referred to herein as “P-glycoprotein”. One known mechanism by which certain drug and multidrug resistance modulators function is by their interaction with P-glycoprotein, which is endogenous in cell membranes, including the membranes of certain drug resistant cells, multidrug resistant tumor cells, gastrointestinal tract cells, and the endothelial cells that form the blood brain barrier. P-glycoprotein acts as an efflux pump for the cell. Certain substances, including treatment drugs for various diseases, are known to be pumped out of the cell by the P-glycoprotein prior to their having an effect on the cell.

ET-743 is known to decrease the rate of mdr1 gene transcription¹⁰. In particular, it is known that 10-50 nM concentrations of the drug inhibit trichostatin-induced transcription of the gene which encodes P-glycoprotein (hereinafter “mdr1”). The concentrations required for inhibition of mdr1 transcription by ET-743 are similar to the concentrations required for its cytotoxic effect. This raises the possibility that the mechanism by which ET-743 inhibits mdr1 transcription may be linked to its cytotoxic properties. Moreover, since P-glycoprotein is responsible for resistance to both drugs and for protection from apoptosis^{12,13}, transcription factors which specifically regulate P-glycoprotein expression may be considered potential targets for the rational design of novel anti-neoplastic agents. While cytotoxic compounds such as ET-743 and the like thus hold considerable promise as antineoplastic agents, their ultimate utility may be limited by, e.g., factors such as difficulty in purification or synthesis in bulk quantities.

The nuclear hormone receptors comprise the largest family of ligand-modulated transcription factors in humans. These receptors mediate the effects of the steroid and thyroid receptors, vitamin D and retinoids. They are intracellular receptors that play important roles in expression of genes involved in physiological processes that include cell growth and differentiation, development, and homeostasis. Upon activation, these receptors are able to regulate expression of genes because they either bind directly to specific DNA sequences called hormone response elements (HREs) or bind indirectly to DNA by binding to other proteins which bind to DNA. Nuclear receptors can be classified based on their DNA binding properties. For example, the glucocorticoid, estrogen, androgen, progestin and mineralocorticoid receptors bind as homodimers to HREs which are organized as inverted repeats. A second class of receptors, including those activated by retinoic acid, thyroid hormone, vitamin D₃, fatty acids/peroxisome proliferators and ecdysone, bind to HREs as heterodimers with a common partner, the retinoid X receptor (i.e., RXR, also known as the 9-cis retinoic acid receptor).

Many of the hormones for the “classical” nuclear receptors were first described at the turn of the last century but in the past decade a larger number of nuclear receptor proteins have been identified that lack known hormones. These proteins have been termed “orphan receptors” and their existence implies that new hormones and signaling molecules which are involved in the regulation of gene expression remain to be identified. Orphan receptors hold considerable promise as they provide the first clues toward the identification of novel regulatory molecules and new drug therapies^{14,15}. Indeed, these proteins have already provided powerful tools for the identification of novel signaling pathways for androstans¹⁶, pregnanes¹⁷ and metabolic signals including fatty acids¹⁸, prostanoids¹⁹, bile acids^{20,22} and cholesterol metabolites^{23,25}. In addition, it has become increasingly clear that orphan receptors are molecular targets for a variety of xenobiotic compounds¹⁵ (e.g., peroxisome proliferators, aminobenzoates²⁶) and pharmaceutical agents (e.g., thiazolidinedione anti-diabetic drugs).

In particular, the orphan receptor SXR (also known as PXR, PAR, PRR and NR112) has been shown to bind to or modulate a broad array of drugs including rifampicin, SR12183, phenobarbital, clotrimazole, RU486, paclitaxel, ritonavir and others^{11,17,26-30,44}. In response to these compounds, SXR activates transcription of cytochrome P450 3A4 (cyp3A4), an enzyme responsible for the metabolic inactivation of approximately 50% of all pharmaceutical agents. Cyp3A4, like mdr1, is a critical gene in the detoxification pathway of xenobiotics. Consistent with their role in detoxification, both CYP3A4 and P-glycoprotein are most highly expressed in the tissues that participate in drug metabolism and elimination, such as liver and intestine^{31,32}. Moreover, many substrates or modulators of CYP3A4 are also substrates or modulators of P-glycoprotein³³. Efficient inducers of CYP3A4, such as rifampicin, phenobarbital, and clotrimazole also activate the transcription of mdr1³⁴. This significant overlap in substrate/inducer specificity suggests that cyp3A4 and mdr1 act in concert to detoxify and deactivate a wide range of compounds since SXR regulates expression of cyp3A4 and mdr1. These findings have led to the suggestion that SXR is a critical sensor in a xenobiotic detoxification system. Thus, SXR mediates the well-established phenomena of auto-induced drug metabolism as well as cross-reactions, whereby one drug promotes the elimination of a co-administered drug. These effects can be a limiting factor in cancer chemotherapy as a variety of anti-neoplastic agents are substrates for CYP3A4 including paclitaxel (Taxol), tamoxifen, mitoxantrone, doxorubicin, cyclophosphamide, ifosfamide and busulphan. Northern blot analysis of SXR revealed that it is abundantly expressed in the liver and small and large intestine. Recent reports suggest SXR is variably expressed in human tumors such as neoplastic breast tissue³⁵.

Nuclear receptors such as SXR thus mediate the transcriptional effects of steroid and related hormones. These receptor proteins have both a conserved DNA-binding domain (DBD) which specifically binds to the DNA at cis-acting elements in their target genes and a ligand binding domain (LBD) which allows for specific activation of the receptor by a particular hormone or other factor. Transcriptional activation of the target gene for a nuclear receptor occurs when the ligand binds to the LBD and induces a conformation change in the receptor that facilitates recruitment of a coactivator or displacement of a corepressor. This results in a receptor complex which can modulate the transcription of the target gene. Recruitment of a coactivator after agonist binding allows the receptor to activate transcription. In contrast, binding of a receptor antagonist to a receptor induces a different conformational change in the receptor such that there is no interaction or there is a non-productive interaction with the transcriptional machinery of the target gene.

It has been determined that hormones are generally small and hydrophobic and are able to diffuse across a plasma membrane and cytoplasm of a cell and bind to nuclear receptors. Upon binding of the hormone to the receptor, the receptor changes its conformation in a manner that activates or suppresses a gene or genes the transcription of which is regulated by the HRE to which the receptor binds. Alternatively, genes can be activated or suppressed by binding of the receptor to other proteins which in turn regulate gene transcription. Examples of such hormones include, steroid hormones, such as testosterone, β-estradiol, aldosterone, cortisol and progesterone, thyroid hormones such as thyroxine (T₄) and triiodothyroxine (T₃) and vitamin D (in vertebrates) along with hormones derived from these. The ability of a low molecular weight, hydrophobic compound such as ET-743 to regulate transcription raises the possibility that ET-743 may act through a ligand-regulated transcription factor.

Due to the implications of the SXR nuclear receptor in modulating drug clearance, there presently exists a further need for compounds and methods for identifying compounds that can provide a pharmacologic intervention in the regulation of transcription of SXR and SXR-regulated genes. Such compounds and methods will be of value to patients who could benefit from modification of SXR-regulated gene transcription and will also be useful as research tools to further elaborate the mechanisms of SXR regulated gene expression.

### SUMMARY OF THE INVENTION

In accordance with the present invention, we have discovered that ET-743 inhibits SXR activation of gene transcription and further that SXR stimulates transcription of the mdr1 gene. The ability of SXR to stimulate mdr1 gene transcription demonstrates the utility of developing cytotoxic drugs such as ET-743 that also inhibit activation of SXR (“SXR-transparent” drugs). Accordingly, the invention provides a method of modulating P-glycoprotein activity which comprises inhibiting trans activation of the mdr1 gene by SXR.

One aspect of the invention is a method for screening compounds to identify antineoplastic agents, which comprises testing said compounds for an ability to inhibit SXR.

A second aspect of the invention is a method of decreasing multidrug resistance in a cell or cells which comprises inhibiting the ability of SXR to trans activate mdr1 gene transcription.

A third aspect of the invention is a method for the treatment or prophylaxis of abnormal cell proliferation in a mammal which comprises administering to such mammal an effective amount of an SXR antagonist, wherein the SXR antagonist decreases the level of mdr1 gene transcription in the tumor cells.

Another aspect of the invention is a method for treating a disorder in a mammal which comprises administering to the mammal an effective amount of a therapeutic agent and inhibiting clearance or breakdown of said therapeutic agent by inhibiting SXR.

A further aspect of the invention is a method of screening compounds for an ability to inhibit trans activation of transcription of an SXR target gene by SXR which comprises determining whether the presence of one or more of said compounds in an assay comprising SXR and said target gene inhibits transcription of said target gene as compared to transcription of said target gene in the absence of said one or more compounds. By said target gene is meant a natural or a synthetic nucleic acid which is responsive to SXR.

Yet another embodiment of the invention is a method of screening compounds for a putative antineoplastic agent which comprises determining whether the presence of one or more of said compounds in an assay comprising SXR and a target gene of SXR inhibits transcription of said target gene as compared to transcription of said target gene in the absence of said one or more compounds.

Another embodiment of the invention is a method to screen compounds for a putative therapeutic agent, comprising:

a) adding an SXR ligand to cells;

b) measuring an activity which is decreased or an amount of a molecule the synthesis of which is decreased by addition of said ligand;

c) adding one or more of said compounds to the cells of step (a) or to cells to which SXR ligand is added;

d) measuring an activity or amount of a molecule as in step (b) for said cells of step (c);

e) determining whether said one or more compounds inhibited the decrease in activity or the decrease in synthesis;

wherein a compound or compounds which inhibit said decrease in activity or said decrease in synthesis of said molecule are putative antineoplastic agents.

The invention also encompasses a method for screening compounds as putative candidates for an ability to decrease catabolism of a drug in a cell or to decrease the ability of a cell to pump said drug out of said cell, said method comprising the steps of determining whether the presence of one or more of said compounds in an assay comprising SXR and said target gene inhibits transcription of said target gene as compared to transcription of said target gene in the absence of said one or more compounds, wherein a compound which inhibits transcription of said target gene is a candidate for decreasing catabolism of a drug or decreasing the ability of a cell to pump said drug out of said cell.

In addition to screening for antagonists which act against agonists and thereby inhibit receptor activation, one aspect of the invention is to screen for inverse agonists. An inverse agonist is a compound which has the opposite effect to an agonist and will block activity. This is well known to those of skill in the art and is illustrated in Picard⁴⁹.

Yet a further aspect of the invention is a method of therapy which comprises coadministering a drug and an agent that modulates the activity or expression of SXR.

Another aspect of the invention is a method of increasing the effectiveness of a drug which comprises coadministering said drug with an agent that modulates the actions of SXR.

The invention also provides a method of inhibiting drug metabolism and/or drug export in a patient receiving treatment with said drug, which method comprises administering to said patient an effective amount of an SXR inhibitor.

The invention is also directed to a process for making a therapeutic composition which comprises the steps of:

a) screening compounds for an ability to inhibit SXR activity;

b) determining which of said compounds inhibit SXR activity;

c) selecting a compound which was determined to inhibit SXR activity;

d) obtaining a therapeutically effective amount of said compound selected according to step (c); and

e) combining a therapeutically effective amount of the selected compound with one or more pharmaceutically acceptable excipients to form a therapeutic composition.

Further aspects of the invention include a therapeutic composition made by the preceding method and methods of inhibiting drug resistance by administering an effective amount of the therapeutic composition.

Yet another aspect of the invention is a method for selecting a compound for use for treating a pathological condition in a mammal wherein said compound is selected by:

a) preparing a system comprising a ligand binding domain of SXR and an SXR target gene wherein an interaction between said ligand binding domain of SXR and said target gene produces a detectable signal;

b) measuring said detectable signal of said system in step (a);

c) adding a compound to a system of step (e);

d) measuring a signal of said system of step (c); and

e) selecting a compound wherein said signal of step (d) is less than said signal of step (b).

The invention also includes compounds selected by the preceding procedure and pharmaceutical compositions comprising the selected compounds.

### BRIEF DESCRIPTION OF THE FIGURES

FIGS. 1A-D demonstrate inhibition of ligand-induced activation of SXR and mdr1 expression by ET-743.

### DETAILED DESCRIPTION OF THE INVENTION

As the first nuclear receptors were cloned nearly fifteen years ago, a large body of biochemical, genetic and structural studies have provided a clear and detailed understanding of how these proteins regulate transcription. The nuclear hormone receptors possess conserved DNA-binding (DBD) and ligand-binding domains (LBD). In the absence of ligand, receptors such as SXR bind to their cognate HRE as an obligate heterodimer with the retinoid X receptor (RXR). In addition, in the absence of ligand, some receptors, including SXR, associate with a co-repressor complex³⁶. This complex contains histone deacetylases which remove acetyl groups from histones and other substrates. Association with the co-repressor complex maintains the DNA transcription machinery in an inactive or repressed state. Transcriptional activation of the target gene occurs when ligand binds to the LBD and induces a conformational change in the SXR which reorients the transcriptional activation domain. This leads to the displacement of the corepressor followed by the recruitment of a coactivator complex, the chromatin is then acetylated and becomes less compact and the rate of transcription is subsequently stimulated.

At least two classes of nuclear receptor coactivators have been identified. The first class includes SRC-1 related proteins (SRC 1, ACTR & GRIP) that modulate chromatin structure by virtue of their histone acetylase activity⁷. A second class includes PBP (also known as DRIP 205 and TRAP 220) which is part of a large transcriptional complex that includes components of the basic transcriptional machinery^{38,39}. Other proteins within each class of nuclear receptor coactivators have been identified and are known to those of skill in the art.

Use of a standard model heterologous cell system to reconstitute SXR-activated transcription allows activity to be monitored in the absence of the metabolic events which may obscure the process being tested. Any suitable heterologous cell system may be used to test the activation of potential or known inhibitors of SXR activation, as long as the cells are capable of being transiently transfected with the appropriate DNA which expresses receptors, reporter genes, response elements, hybrids comprising ligand binding regions, transcriptional activators, corepressors, coactivators and the like. Cells which express one or more of the necessary genes may be used as well. Cell systems that are suitable for the transient expression of mammalian genes and which are amenable to maintenance in culture are well known to those skilled in the art and include, for example, COS or CV-1 cells.

The practice of the present invention employs, unless otherwise indicated, conventional techniques of chemistry, molecular biology, microbiology, recombinant DNA, genetics, immunology, cell biology and cell culture, which are within the skill of the art⁴⁰⁻⁴³. Details of the invention are disclosed in a publication by Synold et al.¹¹, which publication is specifically incorporated herein by reference in its entirety.

To test the inhibition of SXR by ET-743, CV-1 cells were transiently transfected with expression vectors for the receptors along with appropriate reporter constructs according to methods known in the art. The receptors to be tested were expressed in CV-1 cells. Suitable reporter gene constructs are well known to skilled workers in the fields of biochemistry and molecular biology. All transfections additionally contained an expression vector with a cytomegalovirus promoter (pCMV-β-gal) as an internal control. Suitable constructs for use in these studies may conveniently be cloned into a cytomegalovirus expression vector (pCMV). For Example, pCMV-β-gal contains the E. coli β-galactosidase gene expressed under control of the cytomegalovirus promoter/enhancer. Other vectors known in the art can be used in the methods of the present invention.

Genes encoding the following full-length previously described proteins, which are suitable for use in the studies described herein, were cloned into a cytomegalovirus expression vector. All accession numbers in this application refer to GenBank accession numbers. GAL4 fusions containing receptor fragments were constructed by fusing the following protein sequences to the C-terminal end of the yeast GAL4 DNA binding domain (amino acids 1-147) from pSG424⁴⁵: GAL4-SRC1 (human SRC-1, Asp 617 - Asp 769, accession U59302), GAL4-ACTR (human ACTR, Ala 616 - Gln 768, accession AF036892), GAL4-GRIP (mouse GRIP1, Arg 625 - Lys 765, accession U39060), GAL4-PBP (human PBP, Val 574 - Ser 649, accession AF283812), GAL4-SMRT (human SMRT, Arg 1109 - Gly 1330, accession U37146) and GAL4-NCoR (mouse NCoR, Arg 2065 - Gly 2287, accession U35312). VP16 fusions contained the 78 amino acid Herpes virus VP16 transactivation domain (Ala 413 - Gly 490, accession X03141) fused to the N-terminus of the following proteins: VP-SXR (full-length, human SXR, accession AF061056)^{11,16,22,44}.

CMV-β-gal, used as a control gene for comparison with the activation of the receptor or receptor domain being tested, contains the E. coli β-galactosidase coding sequences derived from pCH110 (accession U02445). This gene was conveniently used here, however, any unrelated gene which is available and for which a convenient assay exists to measure its activation may be used as a control with the methods of this invention.

CV-1 cells for the activation assays were grown in Dulbecco's modified Eagle's medium supplemented with 10% resin charcoal-stripped fetal bovine serum, 50 U/ml penicillin G and 50 μg/ml streptomycin sulfate (DMEM-FBS) at 37° C. in 5% CO₂. One day prior to transfection, cells were plated to 50-80% confluence using phenol red free DMEM-FBS.

The cells were transiently transfected by lipofection but other methods of transfection of DNA into cells can be utilized without deviating from the spirit of the invention. Luciferase reporter constructs (300 ng/10⁵ cells) and cytomegalovirus-driven expression vectors (20-50 ng/10⁵ cells) were added, with CMV-μ-gal (500 ng/10⁵ cells) as an internal control. After 2 hours, the liposomes were removed and the cells were treated for approximately 16 hours with phenol red free DMEM-FBS containing the test bile acid and other compounds.

Any compound which is a candidate for inhibition of SXR may be tested by this method. Generally, compounds are tested at several different concentrations. After exposure to ligand, the cells were harvested and assayed for luciferase and β-galactosidase activity (internal control) or activity of any desired reporter gene.

Activity of the reporter gene can be conveniently normalized to the internal control and the data plotted as fold activation relative to untreated cells. Any response element compatible with the assay system may be used. Oligonucleotide sequences which are functionally homologous to the DNA sequence (hormone response elements or HREs) to which the nuclear receptor binds are contemplated for use with the inventive methods. Functionally homologous sequences are sequences which bind the receptor, receptor heterodimer or the indicated DNA binding domain under the conditions of the assay. Functionally homologous sequences are easily determined in an empirical fashion. Response elements can be modified by methods known in the art to increase or decrease the binding of the response element to the nuclear receptor.

We have found that the orphan nuclear receptor SXR can activate transcription of the mdr1 gene. This led us to postulate that the transcriptional inhibitory effects of ET-743 on mdr1 transcription were mediated by SXR. Indeed, ET-743 inhibited SXR at concentrations (IC₅₀=5 nM) that match those required for cytotoxicity. These data provide a link between ET-743 and a molecular target, SXR, that responds to nanomolar concentrations of the drug. In addition, by defining ET-743 as a modulator of SXR activity, these data demonstrate that SXR is a molecular target for high throughput screens aimed at identifying low molecular weight anti-neoplastic agents.

Although ET-743 has considerable promise, its ultimate utility may be limited by the fact that the compound is derived from a marine tunicate (Ecteinascidia turbinata) and the compound has been difficult to purify or synthesize in bulk quantities^{1,46}. Despite such drawbacks, the identification of a molecular target for ET-743, such as SXR, provides a rapid and reliable high-throughput approach for the screening of alternative synthetic or natural product inhibitors of SXR. Finally, just as the screening of breast cancers for estrogen receptor (ER) expression is predictive of a response to the ER antagonist tamoxifen⁴⁷, the identification and validation of SXR as a target of ET-743 can provide a clinical tool to predict the likelihood that an individual tumor will respond to ET-743.

### General Methods

Transient Transfection Assays

CV-1 cells were grown in Dulbecco's Modified Eagle's medium supplemented with 10% resin-charcoal stripped fetal bovine serum, 50 U/ml penicillin G and 50 μg/ml streptomycin sulfate (DMEM-FBS) at 37° C. in 5% CO₂. One day prior to transfection, cells were plated to 50-80% confluence using phenol-red free DMEM-FBS. Cells were transiently transfected by lipofection as described previously⁴⁸. Luciferase reporter constructs (300 ng/10⁵ cells) containing the herpes virus thymidine kinase promoter (−105/+51) linked to the appropriate hormone response element and cytomegalovirus driven expression vectors (20-50 ng/10⁵ cells) were added, along with CMV-p-gal as an internal control. Mammalian expression vectors utilize the cytomegalovirus promoter/enhancer. After incubation with liposomes for 2 hours, the liposomes were removed and cells treated for approximately 16 hours with phenol-red free DMEM-FBS containing an appropriate concentration of agonist or antagonist. After exposure to ligand, the cells were harvested and assayed for luciferase and/or β-galactosidase activity according to known methods.

Human LS 180 cells were maintained in Eagle's minimal essential medium supplemented with 10% fetal bovine serum, 1 mM sodium pyruvate, 2 mM L-glutamine, non-essential amino acids, 50 U/ml penicillin G and 50 g/ml streptomycin sulfate. One day prior to treatment, the LS180 cells were switched to phenol-red free media containing 10% resin-charcoal stripped fetal bovine serum and then treated for an additional 24 hours with the indicated compounds. Northern blots were prepared from total RNA and analyzed with the following probes: mdr1 (accession NM_000927, nucleotides 843-1111), cyp3A4 (accession Ml 8907, nucleotides 1521-2058) and GAPDH (accession NM_002046, nt 101-331) as a control.

The term “functional association” refers to an interaction of two or more proteins or fragments thereof, either in their native state or as part of a hybrid molecule, wherein the interaction as part of a hybrid molecule mimics the association that takes place between such proteins or fragments in vivo or in vitro. The interaction need not be direct contact between the two specific proteins, rather the interaction can be indirect, e.g., the proteins can be part of a complex. In two hybrid transcriptional assays, two proteins or protein fragments functionally associate when one fragment is expressed as a hybrid protein with a DNA binding domain and the other is expressed as a hybrid protein with a transcriptional activator. In this system, functional association of the two protein fragments results in localization of the transcriptional activator to a region of the DNA which is recognized by the DNA binding domain and subsequent expression of a reporter gene that is operatively linked to the DNA binding domain²².

### EXAMPLES

The present invention is further detailed in the following Examples, which are offered by way of illustration and are not intended to limit the invention in any manner. Standard techniques well known in the art or the techniques specifically described herein were utilized.

### Example 1

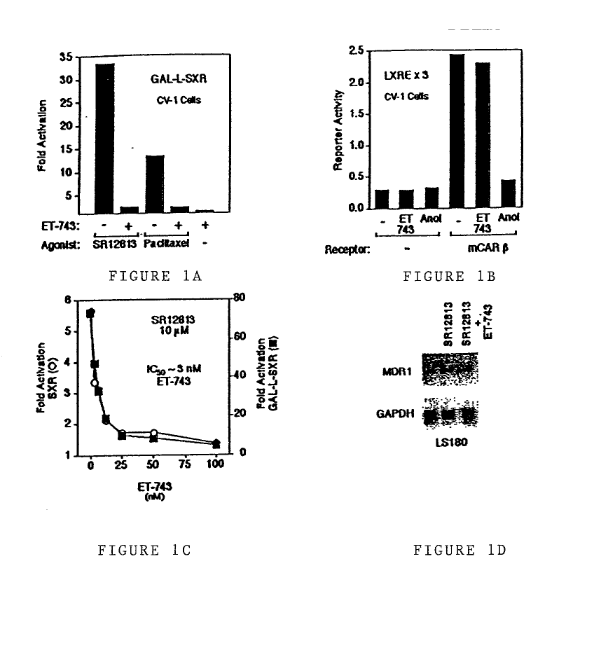

This Example demonstrates Ecteinascidin-743-induced inhibition of ligand-activated SXR. It was previously known that ET-743 is a potent inhibitor of mdr1 transcription¹¹. We therefore postulated that ET-743 may inhibit mdr1 by suppressing SXR activity. FIGS. 1A-D show the results of ET-743 inhibition of ligand-induced activation of SXR and mdr1. Inhibition by 50 nM ET-743 resulted in complete suppression of ligand-activated SXR transcription (FIG. 1A). This effect was specific in that ET-743 had no effect on the basal reporter activity or on unliganded SXR.

To further explore the specificity of this effect, we determined whether ET-743 can inhibit transactivation by the Constitutive Androstane Receptor, CARβ. SXR and CARβ are closely related receptors that share a high degree of sequence similarity in their DNA-binding and ligand-binding domains and have been shown to bind to an overlapping array of response elements and ligands⁴². CARβ displayed strong constitutive activity which was repressed by its inverse agonist androstanol (FIG. 1B). In contrast, ET-743 had no effect on CARP, further indicating that there is specificity to the inhibitory effects of ET-743.

We next determined the IC₅₀ for inhibition by ET-743 and compared this with the reported IC₅₀s for the cytotoxic effects of this drug. Dose response studies (FIG. 1C) using either wild-type or GAL-L-SXR indicated that ET-743 inhibited ligand-activated SXR with an IC₅₀ of 3 nM. Moreover, 20 nM ET-743 was sufficient to suppress SXR-mediated activation of the endogenous mdr1 gene (FIG. 1D). Thus, the effects of ET-743 observed on SXR are well within the range of IC₅₀s reported for the cytotoxic effects of this drug^{2,4}. Thus, unlike the other biochemical events previously linked to ET-743, inhibition of SXR represents the only molecular target to respond to ET-743 at nanomolar concentrations which are sufficient for cell killing.

Previous results have shown that ET-743 inhibits trichostatin induced transcription of mdr1⁹. Trichostatin is an inhibitor of histone deacetylase (HDAC) enzymes that are part of the corepressor complex that interacts with unliganded nuclear receptors. Using mammalian two hybrid assays, we have found that the corepressor SMRT interacts with unliganded SXR and that SXR ligands displace SMRT. Thus, SXR ligands and HDAC inhibitors either displace or inhibit SXR-associated HDAC activity. These observations indicate a unifying mechanism to account for the ability of ET-743 to inhibit mdr1 transcription and SXR activity.

These results demonstrate that ET-743 inhibits ligand-induced activation of SXR. CV-1 cells were transfected and treated with (+) or without (−) ligand and with or without 50 nM ET-743 (FIG. 1A). Reporter gene activity was determined and fold activation was plotted for each treatment. FIG. 1B shows that ET-743 has no effect on CARP. CV-1 cells were transfected with or without an expression vector for CARP and treated either with the CARP antagonist androstanol (5 μM) or with ET-743 (50 nM). FIG. 1C shows the dose response for inhibition of wild-type and GAL-L-SXR by ET-743. Cells were transfected with either wild-type or GAL-L-SXR and their corresponding reporters. After transfection cells were maintained in media or media supplemented with 10 μM SR12813 or SR12813 plus the indicated concentrations of ET-743. FIG. 1D shows the results of Northern blot analysis of LS180 cells treated with the SXR ligand SR12813 +/−20 nM ET-743. As seen in FIG. 1D, ET-743 inhibits SXR-mediated activation of the mdr1 gene.

### Example 2

A mammalian two-hybrid assay was used to determine the effects of the Et-743 analog Pt650 on coregulator recruitment for SXR. CV-1 cells were transfected as indicated above with the indicated hybrid expression vectors and a β-galactosidase vector as an internal control. Reporter activity was measured and normalized to the internal β-galactosidase control and is reported as a proportion of internal β-galactosidase activity. CV- 1 cells were transiently transfected with a GAL4 reporter construct and an expression vector encoding a first hybrid protein which is a DNA transcription activator containing the VP16 transactivation domain linked to the ligand binding domain of SXR (VP-L-SXR). In addition, cells were also transfected with expression vectors encoding the GAL4 DNA binding domain alone or a second hybrid protein which is the GAL4 DNA binding domain linked to the receptor interaction domains of the nuclear receptor coactivators SRC 1, ACTR, GRIP or PBP, or the nuclear receptor corepressors SMRT or NCoR, as indicated. The GAL4 reporter construct comprised four copies of a yeast GAL4 upstream activation sequence operatively linked to the herpes thymidine kinase promoter and the luciferase reporter gene (UASGx4-TK-luc).

After transfection, cells were treated with control media or media containing the indicated SXR agonist ligand or Pt650. PT650 was added at a concentration of 20 nM and each SXR agonist ligand was added at the concentrations indicated. In this system, luciferase reporter expression is activated if the nuclear receptor SXR agonist ligand interacts with the nuclear receptor ligand binding domain of the first hybrid protein, resulting in a conformational change in the nuclear receptor ligand binding domain of the first hybrid and association of the ligand binding domain with the coactivator of the second hybrid. In this system, association of a GALA-coactivator or hybrid with the nuclear receptor ligand binding domain-VP transcriptional activator hybrid results in recruitment of the VP transcriptional activator to GALA DNA binding sequences. Recruitment of the VP transcriptional activator results in transcription and expression of the luciferase gene from the TK promoter of the reporter gene construct.

For corepressors, luciferase reporter expression is activated when the nuclear receptor ligand binding domain of the first hybrid protein interacts with the corepressor of the second hybrid in the absence of agonist ligand. The SXR ligand results in a conformational change in the nuclear receptor ligand binding domain of the first hybrid and inhibits the association of the ligand binding domain with the corepressor of the second hybrid. This results in loss of transcriptional activation of the luciferase gene from the TK promoter of the reporter gene construct.

It will readily be recognized by one skilled in the relevant art that the reporter gene, promoter and transcriptional activator can be replaced in this system without deviating from the current invention. Any reporter gene-promoter-upstream activator construct which will enable detection of functional interaction of nuclear receptor ligand binding domains with coactivator or co-repressor can be utilized.

The results of Example 2 are shown in Table 1, which demonstrates that the ET-743 analog Pt650 displaces coactivator from agonist bound SXR and that it reverses corepressor displacement agonist bound SXR. ET-743 functions in a similar manner. These results demonstrate that the current system can be used to find functional equivalent compounds of Et-743 which can inhibit agonist activated SXR including its ability to displace corepressors and recruit coactivators.

TABLE 1

| | | | Reporter Activity | | | |
|---|---|---|---|---|---|---|
| | | | | | SXR ligand: | |
| | | Ligand-binding | No | Pt650 | SR12813 | SR12813 + |
| Reporter Gene | GAL 4 hybrid | Hybrid | Ligand | 20 nM | (10 μM) | Pt650 |
| UASGx4-TK-luc | GAL4-(no hybrid) | | 0.26 | 0.17 | 0.21 | 0.20 |
| UASGx4-TK-luc | GAL4-hSRC RID 1-3 | VP-L-hSXR | 1.47 | 2.01 | 27.71 | 2.07 |
| UASGx4-TK-luc | GAL4-hACTR RID 1-3 | VP-L-hSXR | 0.23 | 0.22 | 7.53 | 0.47 |
| UASGx4-TK-luc | GAL4-mGRIP 1-3 | VP-L-hSXR | 0.42 | 0.56 | 17.81 | 1.01 |
| UASGx4-TK-luc | GAL4-hPBP RID 1-2 | VP-L-hSXR | 0.96 | 1.50 | 27.39 | 1.60 |
| UASGx4-TK-luc | GAL4-hSMRT 3/6 | VP-L-hSXR | 4.05 | 1.93 | 0.73 | 2.19 |
| UASGx4-TK-Iuc | GAL4-mNCoR 3/6 | VP-L-hSXR | 1.02 | 0.42 | 0.56 | 0.47 |

### LIST OF REFERENCES

1. Rinehart, K. L. Antitumor compounds from tunicates. Med Res Rev 20, 1-27 (2000).

2. Izbicka, E., Lawrence, R., Raymond, E., Eckhardt, G., Faircloth, G., et al. In vitro antitumor activity of the novel marine agent, ecteinascidin-743 (ET-743, NSC-648766) against human tumors explanted from patients. Ann Oncol 9, 981-7 (1998).

3. Hendriks, H. R., Fiebig, H. H., Giavazzi, R., Langdon, S. P., Jimeno, J. M., et al. High antitumour activity of EM43 against human tumour xenografts from melanoma, non-small-cell lung and ovarian cancer. Ann Oncol 10, 1233-40 (1999).

4. Martinez, E. J., Owa, T., Schreiber, S. L. & Corey, E. J. Phthalascidin, a synthetic antitumor agent with potency and mode of action comparable to ecteinascidin 743. Proc Natl Acad Sci USA 96;3496-501 (1999).

5. Zewail-Foote, M. & Hurley, L. H. Ecteinascidin 743: a minor groove alkylator that bends DNA toward the major groove. J Med Chem 42, 2493-7 (1999).

6. Pommier, Y., Kohlhagen, G., Bailly, C., Waring, M., Mazumder, A., et al. DNA sequence- and structure-selective alkylation of guanine N2 in the DNA minor groove by ecteinascidin 743, a potent antitumor compound from the Caribbean tunicate Ecteinascidia turbinata. Biochemistry 35, 13303-9 (1996).

7. Takebayashi, Y., Pourquier, P., Yoshida, A., Kohlhagen, G. & Pommier, Y. Poisoning of human DNA topoisomerase I by ecteinascidin 743, an anticancer drug that selectively alkylates DNA in the minor groove. Proc Natl Acad Sci USA 96, 7196-201 (1999).

8. Bonfanti, M., La Valle, E., Fernandez Sousa Faro, J. M., Faircloth, G., Caretti, G., et al. Effect of ecteinascidin-743 on the interaction between DNA binding proteins and DNA. Anticancer Drug Des 14, 179-86 (1999).

9. Jin, S., Gorfajn, B., Faircloth, G. & Scotto, K. W. Ecteinascidin 743, a transcription-targeted chemotherapeutic that inhibits MDR I activation. Proc Natl Acad Sci USA 97, 6775-9 (2000).

10. Minuzzo, M., Marchini, S., Broggini, M., Faircloth, G., D'Incalci, M., et al. Interference of transcriptional activation by the antineoplastic drug ecteinascidin-743. Proc Natl Acad Sci USA 97, 6780-4 (2000).

11. Synold, T. W., Dussault, I. and Forman, B. M. The orphan nuclear receptor SXR coordinately regulates drug metabolism and efflux. Nat Med 7, 584-590 (2001).

12. Pallis, M. & Russell, N. P-glycoprotein plays a drug-efflux-independent role in augmenting cell survival in acute myeloblastic leukemia and is associated with modulation of a sphingomyelin-ceramide apoptotic pathway. Blood 95, 2897-904 (2000).

13. Thevenod, P., Friedmann, J. M., Katsen, A. D. & Hauser, I. A. Up-regulation of multidrug resistance P-glycoprotein via nuclear factor-kappap activation protects kidney proximal tubule cells from cadmium- and reactive oxygen species-induced apoptosis. J Biol Chem 275, 1887-96 (2000).

14. Kliewer, S. A., Lehmann, J. M. & Willson, T. M. Orphan nuclear receptors: shifting endocrinology into reverse. Science 284, 757-60 (1999).

15. Blumberg, B. & Evans, R. M. Orphan nuclear receptors-new ligands and new possibilities. Genes Dev 12, 3149-55 (1998).

16. Fornan, B. M., Tzameli, I., Choi, H. S., Chen, J., Simha, D., et al. Androstane metabolites bind to and deactivate the nuclear receptor CAR-β. Nature 395, 612-5 (1998).

17. Kliewer, S. A., Moore, J. T., Wade, L., Staudinger, I. L., Watson, M. A., et al. An orphan nuclear receptor activated by pregnanes defines a novel steroid signaling pathway. Cell 92, 73-82 (1998).

18. Forman, B. M., Chen, J. & Evans, R. M. Hypolipidemic drugs, polyunsaturated fatty acids and eicosanoids are ligands for peroxisome proliferator-activated receptors α and δ. Proc Natl Acad Sci USA 94,4312-4317 (1997).

19. Forman, B. M., Tontonoz, P., Chen, J., Brun, R. P., Spiegelman, B. M. and Evans, R. M. 15-Deoxy-δ12, 14-prostaglandin 12 is a ligand for the adipocyte determination factor PPAR gamma. Cell 83, 803-12 (1995).

20. Makishima, M., Okamoto, A. Y., Repa, J. J., Tu, H., Learned, R. M., et al. Identification of a nuclear receptor for bile acids. Science 284, 1362-5 (1999).

21. Parks, D. J., Blanchard, S. G., Bledsoe, R. K., Chandra, G., Consler, T. G., et al. Bile acids: natural ligands for an orphan nuclear receptor. Science 284, 1365-8 (1999).

22. Wang, H., Chen, J., Hollister, K., Sowers, L. C. & Forman, B. M. Endogenous bile acids are ligands for the nuclear receptor FXR/BAR. Mol Cell 3, 543-553 (1999).

23. Fornan, B. M., Ruan, B., Chen, I., Schroepfer, G. J., Jr. & Evans, R. M. The orphan nuclear receptor LXRA is positively and negatively regulated by distinct products of mevalonate metabolism. Proc Natl Acad Sci USA 94, 105 8 8-93 (1997).

24. Janowski, B. A., Willy, P. J., Devi, T. R., Falck, J. R. & Mangelsdorf, D. J. An oxysterol signaling pathway mediated by the nuclear receptor LXRα. Nature 383, 728-31 (1996).

25. Lehmann, J. M., Kliewer, S. A., Moore, L. B., Smith-Oliver, T. A., Oliver, B. B., et al. Activation of nuclear receptor LXR by oxysterols defines a new hormone response pathway. J Biol Chem 272, 3137-40 (1997).

26. Blumberg, B., Sabbagh, W., Jr., Juguilon, H., Bolado, J., Jr., van Meter, C. M., et al. SXR, a novel steroid and xenobiotic-sensing nuclear receptor. Genes Dev 12, 3195-205 (1998).

27. Jones, S. A., Moore, L. B., Shenk, J. L., Wisely, G. B., Hamilton, G. A., et al. The pregnane X receptor: a promiscuous xenobiotic receptor that has diverged during evolution. Mol Endocrinol 14, 27-39 (2000).

28. Zhang, H., LeCulyse, E., Liu, L., Hu, M., Matoney, L., et al. Rat pregnane X receptor: molecular cloning, tissue distribution, and xenobiotic regulation. Arch Biochem Biophys 368, 14-22 (1999).

29. Bertilsson, G., Heidrich, J., Svensson, K., Asman, M., Lendeberg, L., et al. Identification of a human nuclear receptor defines a new signaling pathway for CYP3A induction. Proc Natl Acad Sci USA 95, 12208-13 (1998).

30. Lehmann, J. M., McKee, D. D., Watson, M. A., Willson, T. M., Moore, J. T., et al. The human orphan nuclear receptor PXR is activated by compounds that regulate Cyp3A4 gene expression and cause drug interactions. J Clin Invest 102, 1016-23 (1998).

31. Thiebaut et al., Proc. Natl. Acad. Sci. USA 84:7735-7738, 1987.

32. Watkins et al., J Clin. Invest. 80:1029-1036, 1987.

33. Wacher et al., Mol. Carcinogen. 13:129-134, 1995.

34. Schuetz et al., Mol. Pharmacol. 49:311-318, 1996.

35. Dotzlaw, H., Leygue, E., Watson, P. & Murphy, L. C. The human orphan receptor PXR messenger RNA is expressed in both normal and neoplastic breast tissue. Clin Cancer Res 5, 2103-7 (1999).

36. Glass, C. K. & Rosenfeld, M. G. The coregulator exchange in transcriptional functions of nuclear receptors. Genes Dev 14, 121-41 (2000).

37. Glass, C. K., Rose, D. W. & Rosenfeld, M. G. Nuclear receptor coactivators. Curr Opin Cell Biol 9, 222-32 (1997).

38. Fondell, J. D., Guernah, M., Malik, S. & Roeder, R. G. Thyroid hormone receptor-associated proteins and general positive cofactors mediate thyroid hormone receptor function in the absence of the TATA box-binding protein-associated factors of TFHD. Proc Natl Acad Sci USA 96, 1959-64 (1999).

39. Rachez, C., Lemon, B. D., Suldan, Z., Bromleigh, V., Gamble, M., et al. Ligand-dependent transcription activation by nuclear receptors requires the DRIP complex. Nature 398, 824-8 (1999).

40. Sambrook, J., et al. Molecular Cloning: A Laboratory Manual, 2nd Ed. (Cold Spring Harbor Laboratory, Cold Spring Harbor, N.Y.) 1989; Maniatis. T., et al. Molecular Cloning: A Laboratory Manual (Cold Spring Harbor Laboratory, Cold Spring Harbor, N.Y.) 1982.

41. Ausubel, F. M., et al. Current Protocols in Molecular Biology, (J. Wiley and Sons, N.Y.) 1992.

42. Guthrie, G. and Fink, G. R. Guide to Yeast Genetics and Molecular Biology (Academic Press). 1991.

43. Culture Of Animal Cells (R. I. Freshney, Alan R. Liss, Inc.) 1987.

44. Dussault, I., Lin, M., Hollister, K, Wang, E. H., Synold, T. W. and Forman, B. M. Peptide mimetic HIV protease inhibitors are ligands for the orphan receptor SXR. J Biol Chem (epub ahead of print Jul. 20, 2001).

45. Sadowski, I. and Ptashne, M. A vector for expressing GAL4(1-147) fusions in mammalian cells. Nucl. Acids Res. 17, 7539 (1989)

46. Martinez, E. J. & Corey, E., A new, more efficient, and effective process for the synthesis of a key pentacyclic intermediate for production of ecteinascidin and phthalascidin antitumor agents. Org Lett. 2, 993-6 (2000).

47. Fisher, B., Costantino, J. P., Wickerham, D. L., Redmond, C. K., Kavanah, M., et al. Tamoxifen for prevention of breast cancer: report of the National Surgical Adjuvant Breast and Bowel Project P-1 Study. J Natl Cancer Inst 90, 1371-88 (1998).

48. Forman, B. M., Goode, E., Chen, J. Oro, A. E., Bradley, D. J., Perlmann, T., Noonan, D. J., Burka, L. T., McMorris, T. Lamph, W. W., et al. Identification of a nuclear receptor that is activated by farnesol metabolites. Cell 81, 687-693 (1995).

49. Picard, D. Two orphans find a home. Nature 395:543-544 (1998).

What is claimed is:

1. A method for screening compounds to identify antineoplastic agents, which comprises testing said compounds for an ability to inhibit SXR trans activation of mdr1 gene transcription.

2. A method of decreasing multidrug resistance in a cell or cells which comprises inhibiting the ability of SXR to trans activate mdr1 gene transcription.

3. The method of claim 2, which further comprises contacting the cell or cells with an SXR antagonist, wherein the antagonist inhibits SXR trans activation of mdr1 gene transcription.

4. The method of claim 3 wherein the contact occurs in vivo.

5. A method for the treatment or prophylaxis of abnormal cell proliferation in a mammal which comprises administering to such mammal an effective amount of an SXR antagonist, wherein the SXR antagonist decreases the level of mdr1 gene transcription in the tumor cells.

6. The method of claim 5 wherein the SXR antagonist prevents displacement of an SXR corepressor from SXR.

7. The method of claim 5 wherein the SXR antagonist prevents binding of an SXR ligand to the SXR ligand binding domain.

8. The method of claim 5 wherein the SXR antagonist prevents inhibits interaction between SXR and an SXR coactivator.

9. The method of claim 5 wherein the SXR antagonist is cytotoxic to the cells of the tumor.

10. A method for treating a neoplastic disorder in a mammal which comprises administering to the mammal an antineoplastic effective amount of a cytotoxic agent and inhibiting clearance or breakdown of said cytotoxic agent by inhibiting SXR-mediated transactivation of mdr1.

11. A method of screening compounds for an ability to inhibit trans activation of transcription of an SXR target gene by SXR which comprises determining whether the presence of one or more of said compounds in an assay comprising SXR and said target gene inhibits transcription of said target gene as compared to transcription of said target gene in the absence of said one or more compounds.

12. The method of claim 11 wherein said assay comprises an SXR ligand.

13. The method of claim 12 wherein said ligand is a drug.

14. The method of claim 11 wherein said target gene is mdr1.

15. The method of claim 11 wherein said method is performed in vitro, in vivo or in cells.

16. The method of claim 11 which comprises:
a) adding an SXR ligand to cells;
b) measuring an activity which is increased or an amount of a molecule the synthesis of which is increased by addition of said ligand;
c) adding one or more of said compounds to the cells of step (a) or to cells to which SXR ligand is added;
d) measuring an activity or amount of a molecule as in step (b) for said cells of step (c); and
e) determining whether said one or more of said compounds inhibited the increase in activity or the increase in synthesis of the molecule.

17. The method of claim 16 wherein said molecule is P-glycoprotein.

18. The method of claim 16 wherein said molecule is a gene product of a reporter gene.

19. The method of claim 18 wherein expression of the reporter gene is regulated by the functional association of the ligand binding domain of SXR with an SXR coactivator.

20. The method of claim 19 wherein the SXR coactivator is selected from the group consisting of SRC 1, ACTR, GRIP, PBP, a mimetic peptide which is a coactivator of SXR and a peptide fragment which is a coactivator of SXR.

21. The method of claim 19 wherein expression of the reporter gene is increased by the functional association of the ligand binding domain of SXR with an SXR coactivator.

22. An in vitro method of claim 11 which comprises:
a) mixing SXR and an SXR target gene to form a mixture;
b) measuring an activity which is increased or an amount of a molecule the synthesis of which is increased by addition of a ligand to said mixture;
c) adding one or more of said compounds to the mixture of step (a);
d) measuring an activity or amount of a molecule as in step (b) for said cells of step (c); and
e) determining whether said one or more of said compounds inhibited the increase in activity or the increase in synthesis of the molecule.

23. The method of claim 22 wherein said target gene is mdr1.

24. The method of claim 22 wherein said molecule is P-glycoprotein.

25. The method of claim 22 wherein said ligand is a drug.

26. A method of screening compounds for a putative antineoplastic agent which comprises determining whether the presence of one or more of said compounds in an assay comprising SXR and a target gene of SXR inhibits transcription of said target gene as compared to transcription of said target gene in the absence of said one or more compounds.

27. The method of claim 26 wherein said assay comprises an SXR ligand.

28. The method of claim 27 wherein said ligand is a drug.

29. The method of claim 26 wherein said target gene is mdr1.

30. The method of claim 26 wherein said method is performed in vitro, in vivo or in cells.

31. The method of claim 26, which comprises:
a) adding an SXR ligand to cells;
b) measuring an activity which is increased or an amount of a molecule the synthesis of which is increased by addition of said ligand;
c) adding one or more compounds of said compounds to the cells of step (a) or to cells to which the nuclear ligand is added;
d) measuring an activity or amount of a molecule as in step (b) for said cells of step (c);
e) determining whether said one or more compounds inhibited the increase in activity or the increase in synthesis;
wherein a compound or compounds which inhibit said increase in activity or said increase in synthesis of said molecule are putative antineoplastic agents.

32. The method of claim 31 wherein said molecule is P-glycoprotein.

33. The method of claim 31 wherein said molecule is a gene product of a reporter gene.

34. The method of claim 33 wherein expression of the reporter gene is regulated by the functional association of the ligand binding domain of SXR with an SXR coactivator.

35. The method of claim 24 wherein the SXR coactivator is selected from the group consisting of SRC1, ACTR, GRIP, PBP, a mimetic peptide which is a coactivator of SXR and a peptide fragment which is a coactivator of SXR.

36. The method of claim 34 wherein expression of the reporter gene is increased by the functional association of the ligand binding domain of SXR with an SXR coactivator.

37. An in vitro method of claim 26 which comprises:
a) mixing SXR and an SXR target gene to form a mixture;
b) measuring an activity which is increased or an amount of a molecule the synthesis of which is increased by addition of a ligand to said mixture;
c) adding one or more of said compounds to the mixture of step (a);
d) measuring an activity or amount of a molecule as in step (b) for said cells of step (c); and
e) determining whether said one or more of said compounds inhibited the increase in activity or the increase in synthesis of the molecule.

38. The method of claim 37 wherein said target gene is mdr1.

39. The method of claim 37 wherein said molecule is P-glycoprotein.

40. The method of claim 37 wherein said ligand is a drug.

41. A method to screen compounds for a putative antineoplastic agent, comprising:
a) adding an SXR ligand to cells;
b) measuring an activity which is decreased or an amount of a molecule the synthesis of which is decreased by addition of said ligand;
c) adding one or more of said compounds to the cells of step (a) or to cells to which SXR ligand is added;
d) measuring an activity or amount of a molecule as in step (b) for said cells of step (c);
e) determining whether said one or more compounds inhibited the decrease in activity or the decrease in synthesis;
wherein a compound or compounds which inhibit said decrease in activity or said decrease in synthesis of said molecule are putative antineoplastic agents.

42. The method of claim 41 wherein said molecule is a gene product of a reporter gene.

43. The method of claim 41 wherein said molecule is P-glycoprotein.

44. The method of claim 41 which further comprises administering one of said compounds which inhibits said increase in activity or said increase in synthesis of said molecule to tumor cells and determining if said compound has a cytotoxic effect on the tumor cells.

45. The method of claim 41 which further comprises administering one of said compounds which inhibits said decrease in activity or said decrease in synthesis of said molecule to tumor cells and determining if said compound has a cytotoxic effect on the tumor cells.

46. A method for screening compounds as putative candidates for an ability to decrease catabolism of a drug in a cell or to decrease the ability of a cell to pump said drug out of said cell, said method comprising the steps of determining whether the presence of one or more of said compounds in an assay comprising SXR and said target gene inhibits transcription of said target gene as compared to transcription of said target gene in the absence of said one or more compounds, wherein a compound which inhibits transcription of said target gene is a candidate for decreasing catabolism of a drug or decreasing the ability of a cell to pump said drug out of said cell.

47. The method of claim 46 wherein said assay comprises an SXR ligand.

48. The method of claim 46 wherein said ligand is said drug.

49. The method of claim 46 wherein said target gene is mdr1.

50. The method of claim 46 wherein said method is performed in vitro, in vivo or in cells.

51. The method of claim 46 which comprises:
a) adding an SXR ligand to cells;
b) measuring an activity which is increased or an amount of a molecule the synthesis of which is increased by addition of said ligand;
c) adding one or more of said compounds to the cells of step (a) or to cells to which SXR ligand is added;
d) measuring an activity or amount of a molecule as in step (b) for said cells of step (c); and
e) determining whether said one or more of said compounds inhibited the increase in activity or the increase in synthesis of the molecule.

52. The method of claim 51 wherein said molecule is P-glycoprotein.

53. The method of claim 51 wherein said molecule is a gene product of a reporter gene.

54. The method of claim 53 wherein expression of the reporter gene is regulated by the functional association of the ligand binding domain of SXR with an SXR coactivator.

55. The method of claim 54 wherein the SXR coactivator is selected from the group consisting of SRC1, ACTR, GRIP, PBP, a mimetic peptide which is a coactivator of SXR and a peptide fragment which is a coactivator of SXR.

56. The method of claim 54 wherein expression of the reporter gene is increased by the functional association of the ligand binding domain of SXR with an SXR coactivator.

57. An in vitro method of claim 46 which comprises:
a) mixing SXR and an SXR target gene to form a mixture;
b) measuring an activity which is increased or an amount of a molecule the synthesis of which is increased by addition of a ligand to said mixture;
c) adding one or more of said compounds to the mixture of step (a);
d) measuring an activity or amount of a molecule as in step (b) for said cells of step (c); and
e) determining whether said one or more of said compounds inhibited the increase in activity or the increase in synthesis of the molecule.

58. The method of claim 57 wherein said target gene is mdr1.

59. The method of claim 57 wherein said molecule is P-glycoprotein.

60. The method of claim 57 wherein said ligand is a drug.

61. A method of drug chemotherapy which comprises coadministering a drug and an agent that modulates the activity or expression of SXR.

62. A method of claim 61 which comprises coadministering a drug and an agent that downregulates the activity or expression of SXR.

63. A method of claim 61 which comprises coadministering a drug and an agent that upregulates the activity or expression of SXR.

64. A method of increasing the effectiveness of a drug which comprises coadministering said drug with an agent that modulates the actions of SXR.

65. A method of claim 61 wherein said agent is an SXR antagonist.

66. A method of claim 61 wherein said agent is an SXR agonist.

67. A method of inhibiting drug metabolism in a patient receiving treatment with said drug, which method comprises administering to said patient an effective amount of an SXR inhibitor.

68. A process for making a therapeutic composition which comprises the steps of:
a) screening compounds for an ability to inhibit SXR activity or to inhibit transcription or translation of SXR;
b) determining which of said compounds inhibit SXR activity or inhibit transcription or translation of SXR;
c) selecting a compound which was determined to inhibit SXR activity or to inhibit transcription or translation of SXR;
d) obtaining a therapeutically effective amount of said compound selected according to step (c); and
e) combining a therapeutically effective amount of the selected compound with one or more pharmaceutically acceptable excipients to form a therapeutic composition.

69. The method of claim 68 wherein said screening comprises the steps of claim 11.

70. The method of claim 68 wherein said screening comprises the steps of claim 26.

71. A therapeutic composition made by the process of claim 68.

72. A method of inhibiting drug resistance by administering an effective amount of a therapeutic composition of claim 71 which modulates SXR activity or SXR expression.

73. A method for selecting a compound for use for treating a pathological condition in a mammal wherein said compound is selected by:
a) preparing a system comprising a ligand binding domain of SXR and an SXR target gene wherein an interaction between said ligand binding domain of SXR and said target gene produces a detectable signal;
b) measuring said detectable signal of said system in step (a);
c) adding a compound to a system of step (e);
d) measuring a signal of said system of step (c); and
e) selecting a compound wherein said signal of step (d) is less than said signal of step (b).

74. The method of claim 73 wherein said interaction is a direct interaction.

75. The method of claim 73 wherein said interaction is an indirect interaction.

76. The method of claim 73 wherein said pathological condition is a cancer.

77. The method of claim 73 wherein said target gene is mdr1.

78. The method of claim 73 wherein said detectable signal is mdr1 RNA.

79. The method of claim 73 wherein said detectable signal is P-glycoprotein.

80. The method of claim 73 wherein said system comprises a cell.

81. The method of claim 80 wherein said cell comprises a vector comprising said target gene.

82. The method of claim 73 wherein said system comprises a ligand that binds to said ligand binding domain of SXR under physiological conditions.

83. The method of claim 82 wherein said ligand is a drug or drug candidate.

84. The method of claim 83 wherein said drug or drug candidate is to treat cancer.

85. The method of claim 73 wherein said system comprises components of a two-hybrid assay.

86. The method claim 85 wherein said system comprises a vector encoding an SXR ligand binding domain fused to a peptide which activates said SXR ligand binding domain.

87. The method of claim 85 wherein said peptide is VP16.

88. The method of claim 85 wherein said system comprises a vector encoding a signal generating enzyme.

89. The method of claim 73 wherein said method is performed in vitro.

90. The method of claim 89 wherein said system comprises a ligand binding domain of SXR and said target gene.

91. The method of claim 90 wherein said target gene is mdr1.

92. A compound for treating a pathological condition in a mammal wherein said compound is selected by the method of claim 73.

93. A pharmaceutical composition comprising said compound of claim 92.
